(19) United States
(12) Patent Application Publication
(10) Pub. No.: US 2015/0011654 A1
(43) Pub. Date: Jan. 8, 2015

(54) STABLE WATER-IN-OIL EMULSIONS WITH HLB-TYPE EMULSIFIERS

(71) Applicant: BEIERSDORF AG, Hamburg (DE)

(72) Inventors: Stephanie Von Der Fecht, Wedel (DE); Svenja Lena Moellgaard, Hamburg (DE); Isabel Balcke, Hamburg (DE); Petra Koch, Hamburg (DE)

(21) Appl. No.: 14/376,981

(22) PCT Filed: Feb. 12, 2013
(86) PCT No.: PCT/EP2013/052731

§ 371 Date: Aug. 6, 2014

(30) Foreign Application Priority Data
Feb. 16, 2012 (DE) 102012002950.0

(51) Int. Cl. A61K 8/06; A61K 8/34; A61K 8/86; A61K 8/73; A61K 8/25; A61Q 19/00; A61K 8/36

(52) U.S. Cl. CPC A61K 8/064; A61Q 19/007; A61K 8/345; A61K 8/361; A61K 8/732; A61K 8/25; A61K 8/86; A61K 2800/30; A61K 2800/594; A61K 2800/52
USPC 514/738; 514/785

(57) ABSTRACT

Cosmetic and/or dermatological preparations based on a water-in-oil emulsion comprising at least two W/O emulsifiers which differ in their HLB value up to a maximum of 1 lead to sensorily acceptable preparations and at the same stable W/O emulsions.

The invention comprises cosmetic and/or dermatological preparation based on a water-in-oil emulsion comprising at least two W/O emulsifiers which differ in their HLB value by at most 1.

As cosmetic or medicinal preparations often emulsions, in particular W/O, O/W, O/W/O or W/O/W emulsions, are used. In general, emulsions are understood as meaning heterogeneous systems which consist of two liquids which are immiscible or have only limited miscibility with one another and are usually referred to as phases. In an emulsion, one of the two liquids (W/O) is dispersed in the form of very fine droplets in the other liquid. The liquids (pure or as solutions) are present in an emulsion in a more or less fine distribution, which is generally of only limited stability.

If the two liquids are water and oil and oil droplets are present in finely distributed form in water, then this is an oil-in-water emulsion (O/W emulsion, e.g. milk). The basic character, for example electrical conductivity, sensory properties, ability of the continuous phase to be stained, of an O/W emulsion is determined by the water. In the case of a water-in-oil emulsion (W/O emulsion, e.g. butter), the principle is reversed, with the basic character here being determined by the oil.

The prior art recognizes several essential factors which have a positive influence on the stability and rheology of emulsions.

For their formation and stabilization, emulsions generally require one or more emulsifiers, thickeners and/or consistency regulators in order to be stable over a cosmetically acceptable period, generally 1 year after opening a cosmetic preparation.

A particular challenge is the formulation of flowable emulsions. On account of their pleasant spreadability these are highly valued by the consumer; however, the stable formulation is a technological challenge.

To stabilize emulsions, ethoxylated emulsifiers are often used which, as is generally known, lead to resilient, stable emulsion preparations and can often cover a relatively wide sensory range. However, it is known that ethoxylated emulsifiers act as penetration enhancers, due to their PEG units.

For example, EP 1 192 935 A2 discloses W/O emulsions comprising polyethers such as PEG-45 (dodecyl glycol) copolymer and PEG-22 (dodecyl glycol) copolymer.

The use of polyethylene glycols and/or polyethylene glycol derivatives and modifications thereof are a topic of controversial discussion amongst the public since they are suspected, following topical application, of making the skin more permeable to foreign bodies such as e.g. harmful substances.

Furthermore, under the action of solar radiation, the photo-unstable polyethylene glycol-containing (PEG) emulsifiers can be decomposed and trigger unsightly skin reactions.

For the reasons specified, cosmetic formulations are increasingly being sought by the consumer which are free from this class of substance.

Also, in order to be able to develop W/O emulsions that are sensorily pleasant, in most cases PEG emulsifiers are currently used. Since many consumers are looking for products which are free from this class of substance, it is a technological challenge to stabilize emulsions without these substances and at the same time to achieve a pleasant skin feel.

As regards the stability, the challenge is in particular the scaling-up and, in the case of skin feel, parameters such as “rapid and easy absorption, low stickiness” are difficult to achieve without PEG stabilizers.

It is therefore desirable to provide emulsion preparations without ethoxylated emulsifiers which nevertheless can be varied as widely as possible and are above all else stable emulsions.

Furthermore, cosmetic or dermatological preparations have to satisfy a number of esthetic and sensory aspects in order to achieve adequate consumer acceptance.

A cosmetic preparation “Bebe Zartpflege Zartcreme” with the ingredients methylglucose isostearate, hydrogenated castor oil and diisostearoyl polyglyceryl-3 dimer dilinoleate is on the market.

DE 60 2004 013 358 T2 discloses multiphase emulsions. The described emulsions are “multiple water-in-oil-in-water emulsions”, i.e. W/O/W systems. Moreover, polyglyceryl-3 diisostearate is mentioned as W/O emulsifier.

DE 10 2008 028 822 A1 discloses an extensive reference work on emulsifiers and oils. Specifically, cosmetic stick compositions in the form of oil-in-water dispersions/emulsions are described.

DE 199 24 277 A1 describes W/O emulsions and their known advantages and disadvantages. The disadvantages are said to be overcome by using interface-active substances from the group of alkylmethicone copolyols.

US 20030103921 A1 discloses antiperspirant-containing microemulsions.

DE 2830492 A1 and US 20090285876 A1 do not disclose any W/O emulsions which are stabilized with the help of two W/O emulsifiers.

WO 2009/080657 A2 discloses W/O emulsions comprising, besides hydrophobically modified polysaccharides, starches and/or agar polyglycerol-4 diisostearate polyhydroxystearate sebacate (Isolan GPS®).

FR 2927535 discloses stable water-in-oil emulsions as cosmetic preparations comprising esters of fatty acids and polyols. A preferred ester that is given is, amongst others, polyglycerol-4 diisostearate polyhydroxystearate sebacate (Isolan GPS®).

WO 2008/055692 A2 describes silicone-free skin protectants comprising, besides oils and polyols, inter alia as emulsifiers polyglycerol-4 diisostearate polyhydroxystearate sebacate (GPS) and/or polyglyceryl-2 dipolyhydroxystearate (PGPH).

The use of this emulsifier combination leads either to non-flowable, but instead stable formulations or to cosmetic preparations which, upon storage at 40 degrees Celsius, exhibit oil separation after a certain amount of time. Moreover, the emulsions stabilized by means of polyglycerol-4 diisostearate polyhydroxystearate sebacate and/or polyglyceryl-2 dipolyhydroxystearate are characterized by sticky, oily sensorics. If readily spreading oils are used for optimizing these undesired sensorics, then this likewise results in a stability loss upon longer-term storage at 40° C.

The invention is a cosmetic and/or dermatological preparation based on a water-in-oil emulsion comprising at least two W/O emulsifiers. Two of the W/O emulsifiers differ in their HLB value by at most 1. Preferred is the combination of emulsifiers with an only small HLB difference of e.g. 0.5 or even the same HLB value. Particular preference is given to using exclusively two emulsifiers.

Preferred W/O emulsifiers selected are diisostearoyl polyglyceryl-3 dimer dilinoleate and polyglyceryl-4 diisostearate/polyhydroxystearate/sebacate. Diisostearoyl polyglyceryl-3 dimer dilinoleates are known as PEG-free emulsifiers as Isolan® PDI and polyglyceryl-4 diisostearate/polyhydroxystearate/sebacates are known as Isolan® GPS from Evonik with an HLB value of about 5.

Surprisingly, by combining two W/O emulsifiers according to the invention, it has been possible to solve the specified technical challenges.

The preparations according to the invention preferably comprise no further additional W/O emulsifiers. The fraction of additional emulsifiers should thus preferably be less than 0.01% by weight, based on the total mass of the preparation, in order to be deemed in accordance with the invention—without additional emulsifier.

In the same way, it is possible according to the invention to dispense with the addition of polyethylene glycols and/or polyethylene glycol derivatives. The fraction of PEGs is therefore less than 1% by weight, in particular 0% by weight, based on the total mass of the preparation.

According to the invention, no further W/O emulsifiers are required for stabilization. Hydrophilic stabilizers such as thickeners and fatty alcohols can optionally be present according to the invention.

The fraction of the W/O emulsifiers according to the invention is preferably in the range from 1 to 5.5% by weight, preferably 1.5 to 3% by weight, particularly preferably 2 to 2.6% by weight, based on the total mass of the preparation.

The fraction of polyglyceryl-4 diisostearate/polyhydroxystearate/sebacate is chosen advantageously in the range from 0.1 to 2.5% by weight, preferably 0.3 to 1.5% by weight, particularly preferably 0.5 to 1% by weight, and the fraction of diisostearoyl polyglyceryl-3 dimer dilinoleate is chosen in the range 0.1 to 3% by weight, preferably 0.5 to 2% by weight, particularly preferably 1 to 1.8% by weight, based on the total mass of the preparation.

The preparations according to the invention advantageously comprise at least one skin moisturizer in a fraction of in total 5 to 20% by weight, based on the total mass of the preparation. Preference is given to a glycerol content of 7 to 15% by weight, particularly preferably of 10% by weight.

By using the emulsifier combination according to the invention, it has been possible to formulate a W/O emulsion which, despite large amounts of skin moisturizers such as glycerol, has a very pleasant skin feel.

Furthermore, the preparations according to the invention are stable in stress tests, such as e.g. storage at elevated and/or fluctuating temperatures. This stability exists not only for the 1 to 5 kg batches produced on the laboratory scale, but also for larger amounts such as e.g. 100 to 500 kg and even during production on the ton-scale. This process, known as “up-scaling”, is known to be particularly sensitive for customary W/O emulsions. For the preparations described in the context of this invention, the up-scaling process has proven to be surprisingly problem-free, which is based on the emulsifier selection according to the invention.

Emulsifiers make it possible for two immiscible liquids (for example oil in water) to be able to combine to give an emulsion. On account of the amphiphilic character, they penetrate with their fat-soluble part into the oil. By virtue of the hydrophilic part, the oil droplet now produced by stirring can be dispersed in the aqueous environment. Emulsifiers have primarily no detersive, surfactant character. Emulsifiers reduce the interfacial tension between the two phases and, besides reducing the interfacial work, also achieve a stabilization of the emulsion formed. They stabilize the formed emulsion by means of interfacial films, as well as by forming steric or electrical barriers, as a result of which the merging (coalescence) of the emulsified particles is prevented.

In order that compounds can be effective as emulsifiers, they must have a certain molecular structure. A structural characteristic of such compounds is their amphiphilic molecular structure. The molecule of such a compound has at least one group with affinity to substances of strong polarity (polar groups) and at least one group with affinity to nonpolar substances (apolar groups).

A distinction is made here between nonionic, anionic and cationic emulsifiers. One characteristic of the hydrophilicity of a given emulsifier is its HLB value, which is given by the following formula: HLB=20×(1−Mₗᵢₚₒₚₕᵢₗ/M), where Mₗᵢₚₒₚₕᵢₗ stands for the molar mass of the lipophilic fraction in the emulsifier and M stands for the molar mass of the total emulsifier.

In general, emulsifiers with an HLB value up to about 8 are considered to be W/O emulsifiers. By contrast, O/W emulsifiers have HLB values of greater than 8 to 15. Substances with HLB values greater than 15 are often referred to as solubility promoters.

According to the invention, it has now been found that when choosing two W/O emulsifiers which both have an HLB value <8 and both have a similar HLB value, it is possible to formulate cosmetic preparations which are flowable and very pleasant in their sensory appearance, i.e. are not sticky, not oily, and bring about a soft skin feel after spreading and nevertheless have good stability even upon storage at different temperatures. The problem-free up-scaling process is particularly surprising.

The combination of two W/O emulsifiers which differ in their HLB value by at most 1 can therefore be used for producing cosmetic water-in-oil emulsions with improved stability and/or sensorics.

In comparative experiments, the stability and sensorics of preparations according to the invention were investigated with those of the prior art (WO 2008/055692 A2, see Table 1).

TABLE 1
Stability and sensorics of W/O emulsions

| Experiment | | | |
|---|---|---|---|
| number | Emulsifier | Stability | Sensorics |
| 18 | 1.5% PGPH | (v) oil separation | Residue oily, |
| | | | sticky |
| 19 | 2% PGPH | v | Light sensorics, |
| | | | residue somewhat |
| | | | oily, shiny |
| 20 | 2.5% PGPH | oil separation | |
| 21 | 3% PGPH | oil separation | Barely any and matt |
| | | | residue, very pleasant, |
| | | | nonoily, stickier than |
| | | | original |

The preparations with only one W/O emulsifier with an HLB value of 5 (PGPH-polyglyceryl-2 dipolyhydroxystearate) exhibit inadequate stabilities (oil separations) and/or unpleasant sensorics, as shown in Table 1 above.

By contrast, the preparations according to the invention with two W/O emulsifiers which differ merely in their HLB value by at most 1 lead to a sensorily acceptable preparation as well as to stable W/O emulsions (Example 4).

TABLE 2
Stability and sensorics of preparation according to
the invention compared to preparations without emulsifier
combination according to the invention.

| | | Stability under | |
|---|---|---|---|
| | | fluctuating | |
| | Viscosity | temperatures | |
| | in mPas | (−12 to +60° C.) | Sensorics |
| Ex. formulation 4 | 5400 | in order | Easily spreadable |
| | | | with pleasant, |
| | | | care residue |
| Formulation 4 | 3700 | in order | Slippery upon |
| without PDI, | | | spreading, more |
| instead with | | | oily than Ex. 4 |
| 2.2% GPS | | | |
| Formulation 4 without | 3200 | 2 mm oil | Upon spreading |
| PDI and without GPS, | | separation | very light, |
| instead with 2.2% | | | very oily |
| PEG-40 sorbitan | | | |
| perisostearate | | | |

PDI = Diisostearoyl Polyglyceryl-3 Dimer Dilinoleate
GPS = Polyglyceryl-4 Diisostearate/Polyhydroxystearate/Sebacate

It was found that the combination of two W/O emulsifiers which differ only in their HLB value by at most 1 lead to preparations which are both stable and sensorily more pleasant than without the combination.

The investigations also show that just one emulsifier is inadequate for achieving the desired properties, such as improved sensorics and stability.

Rotary rheometers, among others, are available for measuring the viscosity of cosmetic preparations.

Measuring the viscosity using a rotary rheometer is performed by rotating a measuring body in the substance to be measured. The viscosity of the substance behaves proportionally to the force with which it counteracts the rotation of the measuring body. This is valid under the prerequisite that measuring body geometry and rotational speed remain the same.

In addition, there are very different measuring body geometries
coaxial cylinder as per DIN 53 019
cone-plate as per DIN 53 019
anchor stirrer or T-spindles or
plate-like measuring bodies as per ISO 2555

The viscosity η is defined as the ratio of shear stress τ and

$$\frac{.}{γ}$$

shear rate

$$η = \frac{τ}{\dot{γ}}$$

The viscosity changes with the shear rate. Consequently, during the processing, discharging or use of the products, it must be noted that the product can behave significantly differently during pumping than during pouring.

The viscosity should thus be ascertained in each case in the shear rate range in which the application lies, i.e. it is necessary to use different rheological investigative methods if it is desired to establish whether the surface of the cream runs quickly in the container, whether the cream is pleasant to apply and absorbed into the skin or whether one wishes to calculate what pumping power is required in order to convey it through a pipeline system.

Expediently and in the context of the present invention, unless stated otherwise, the viscosity η is measured using the instrument “Viscotester VT-02” from Haake at a temperature T=25° C. and the shear rate 10 s⁻¹.

Moreover, it was surprising that according to the invention it was possible to develop a sensorily equivalent or improved preparation which is PEG-free, comprises more than twice as much glycerol and is good in upscaling.

The invention is therefore advantageously a cosmetic or dermatological preparation based on a water-in-oil emulsion and it has in particular no (0%) ethoxylated emulsifiers, polyethylene glycols and/or polyethylene glycol derivatives.

The preparations according to the invention advantageously comprise at least 10 to 30% by weight, preferably 15 to 25% by weight, particularly preferably 18 to 22% by weight, of lipids.

The lipid phase or oil phase does not include the emulsifiers. Advantageously, the oil phase can be composed of at room temperature (RT) solid or semisolid raw materials and liquid raw materials. The fraction of solid or semisolid constituents, based on the oil phase, is advantageously about 40% to 0.1%, preferably about 30 to 3% and particularly preferably about 15 to 5% by weight, based on the total mass of the oil phase.

Preferred lipid components liquid at RT are selected from the group paraffinum liquidum, isopropyl palmitate, C13-16 isoparaffin and natural oils such as argan oil, olive oil, sunflower oil or almond oil.

Preferred lipid components solid or semisolid at room temperature (RT) are selected from the group cera microcristallina, cetyl palmitate and/or Vaseline (cera microcristallina+paraffinum liquidum).

The cosmetic and/or dermatological preparation according to the invention is a water-in-oil emulsion that is flowable at room temperature and is not a multiple emulsion such as W/O/W or O/W/O emulsion. The emulsion according to the invention is likewise advantageously not a microemulsion or nanoemulsion.

The preparations according to the invention are advantageously flowable at room temperature (20° C.).

Furthermore, the preparations according to the invention advantageously comprise one or more powder raw materials, which are present preferably in a fraction of up to 5% by weight, preferably 0.2 to 2% by weight, based on the total mass of the preparation. Preferred powder raw materials are aluminum starch octenylsuccinate and/or talc.

The cosmetic or dermatological preparations according to the invention can also comprise cosmetic auxiliaries and further active ingredients as are customarily used in such preparations, e.g. preservatives, preservation auxiliaries, bactericides, substances for preventing foaming, dyes and colored pigments, thickeners, moisturizing and/or humectant substances, fats, oils, waxes or other customary constituents of a cosmetic or dermatological formulation such as alcohols, polyols, polymers, foam stabilizers, electrolytes, organic solvents or silicone derivatives, self-tanning agents, buffers, pH regulators, plant extracts, surfactants, propellant gases, powders, sebum-absorbing substances, UV filters, active ingredients such as for example anti-aging, anti-cellulite, anti-acne, anti-rosacea, anti-neurodermatitis, antioxidants, moisturizers, chelating agents, antiperspirants, bleaches and colorants etc., provided the addition does not hinder the required properties as regards freedom from PEG, emulsifier content, required stability and sensorics.

The water content of the preparations according to the invention is advantageously between 40 and 80% by weight, preferably between 50% by weight and 70% by weight, particularly preferably between 55% by weight and 65% by weight, in each case based on the total mass of the preparations.

In the case of restrictions to preferably mentioned substances, whether lipids, the W/O emulsifiers or further preferably mentioned constituents, their preferred fraction ranges then also refer to the individual constituents then selected. The other constituents excluded by the restriction then no longer add to the listed fraction ranges.

The examples below illustrate the preparations according to the invention. The numerical values refer to the weight fractions with regard to the total mass of the preparation, unless stated otherwise.

### EXAMPLE 1

0.5% Polyglyceryl-4 Diisostearate/Polyhydroxystearate/Sebacate

1.9% Diisostearoyl Polyglyceryl-3 Dimer Dilinoleate

3% Cera Microcristallina

10% Caprylic/Capric Triglyceride

10% Dicaprylyl Ether

1% Talc

12% Glycerol

1% Propylene glycol

0.1% Hexanediol

0.15% Potassium Sorbate

0.1% Citric Acid

0.2% Sodium Citrate

0.4% Perfume

with Aqua ad 100%

### EXAMPLE 2

0.8% Polyglyceryl-4 Diisostearate/Polyhydroxystearate/Sebacate

1.9% Diisostearoyl Polyglyceryl-3 Dimer Dilinoleate

2.5% Cetyl Palmitate

0.1% Cera Microcristallina

4% C13-16 Isoparaffin

4% Argania Spinosa Kernel Oil

6.5% Paraffinum Liquidum

6.5% Isopropyl Palmitate

10% Glycerol

2% Tapioca Starch

0.15% Potassium Sorbate

0.2% Sodium Citrate

0.1% Citric Acid

0.15% Perfume

with Aqua ad 100%

### EXAMPLE 3

1.5% Polyglyceryl-4 Diisostearate/Polyhydroxystearate/Sebacate

1.5% Diisostearoyl Polyglyceryl-3 Dimer Dilinoleate

2.7% Shea Butter

7.5% Isohexadecane

5.3% Dibutyl Adipate

6.5% Paraffinum Liquidum

0.1% Sunflower oil

13.75% Glycerol

0.5% Ethylhexylglycerin

1% Aluminum Starch Octenylsuccinate

1% Nylon-12

0.15% Potassium Sorbate

0.2% Sodium Citrate

0.1% Citric Acid

0.3% Perfume

with Aqua ad 100%

### EXAMPLE 4

0.8% Polyglyceryl-4 Diisostearate/Polyhydroxystearate/Sebacate

1.4% Diisostearoyl Polyglyceryl-3 Dimer Dilinoleate

1% Cera Microcristallina+Paraffinum Liquidum

0.5% Cera Microcristallina

2% Paraffinum Liquidum

1% Prunus Amygdalus Dulcis Oil

0.5% Cetyl Palmitate

9.5% Isopropyl Palmitate

6% C13-16 Isoparaffin

0.5% Aluminum Starch Octenylsuccinate

10% Glycerol

0.15% Potassium Sorbate

0.7% Magnesium Sulfate

0.1% Maris Sal

0.5% Glyceryl Glucoside

0.2% Sodium Citrate

0.1% Citric Acid

0.35% Perfume

with Aqua ad 100%

### EXAMPLE 5

1.4% Polyglyceryl-4 Diisostearate/Polyhydroxystearate/Sebacate

0.8% Diisostearoyl Polyglyceryl-3 Dimer Dilinoleate

0.2% Cera Microcristallina

1% Shea Butter

2.5% Cera Microcristallina+Paraffinum Liquidum

7.5% Caprylic/Capric Triglyceride

12% Dicaprylyl Ether

0.1% Olive oil

1% Talc

0.2% Aluminum Starch Octenylsuccinate

12% Glycerol

0.1% Caprylyl Glycol

0.15% Potassium Sorbate

0.1% Citric Acid

0.2% Sodium Citrate

0.4% Perfume

with Aqua ad 100%

### EXAMPLE 6

1.1% Polyglyceryl-4 Diisostearate/Polyhydroxystearate/Sebacate

1.2% Diisostearoyl Polyglyceryl-3 Dimer Dilinoleate

0.2% Cera Microcristallina

1% Shea Butter

2.5% Cera Microcristallina+Paraffinum Liquidum

7.5% C13-16 Isoparaffin

12% Dicaprylyl Ether

0.1% Olive oil

1% Talc

0.2% Aluminum Starch Octenylsuccinate

12% Glycerol

0.1% Caprylyl Glycol

0.15% Potassium Sorbate

0.1% Citric Acid

0.2% Sodium Citrate

0.4% Perfume

with Aqua ad 100%

1-17. (canceled)

18. A cosmetic and/or dermatological preparation, wherein the preparation is based on a water-in-oil emulsion comprising at least two W/O emulsifiers which differ in their HLB value by at most 1.

19. The preparation of claim 18, wherein the at least two W/O emulsifiers differ in their HLB value by at most 0.5.

20. The preparation of claim 18, wherein only two W/O emulsifiers are present.

21. The preparation of claim 18, wherein the at least two W/O emulsifiers comprise diisostearoyl polyglyceryl-3 dimer dilinoleate and polyglyceryl-4 diisostearate/polyhydroxystearate/sebacate.

22. The preparation of claim 18, wherein the preparation comprises from 1% to 5.5% by weight of W/O emulsifiers, based on a total weight of the preparation.

23. The preparation of claim 22, wherein the preparation comprises from 1.5% to 3% by weight of W/O emulsifiers.

24. The preparation of claim 22, wherein the preparation comprises from 2% to 2.6% by weight of W/O emulsifiers.

25. The preparation of claim 21, wherein the preparation comprises from 0.1% to 2.5% by weight of polyglyceryl-4 diisostearate/polyhydroxystearate/sebacate and from 0.1% to 3% by weight of diisostearoyl polyglyceryl-3 dimer dilinoleate, each based on a total weight of the preparation.

26. The preparation of claim 25, wherein the preparation comprises from 0.3% to 1.5% by weight of polyglyceryl-4 diisostearate/polyhydroxystearate/sebacate and from 0.5% to 2% by weight of diisostearoyl polyglyceryl-3 dimer dilinoleate.

27. The preparation of claim 18, wherein the preparation further comprises a total of from 5% to 20% by weight, based on a total weight of the preparation, of at least one skin moisturizer.

28. The preparation of claim 27, wherein the at least one skin moisturizer comprises glycerol.

29. The preparation of claim 18, wherein the preparation further comprises from 10% to 30% by weight of one or more lipids, based on a total weight of the preparation.

30. The preparation of claim 18, wherein the one or more lipids are selected from paraffinum liquidum, isopropyl palmitate, C13-16 isoparaffin, cera microcristallina, cetyl palmitate, Vaseline (cera microcristallina+paraffinum liquidum) and natural oils.

31. The preparation of claim 18, wherein the preparation further comprises one or more powder raw materials.

32. The preparation of claim 31, wherein the one or more powder raw materials are present in a concentration of up to 5% by weight, based on a total weight of the preparation.

33. The preparation of claim 31, wherein the one or more powder raw materials comprise at least one of aluminum starch octenylsuccinate and talc.

34. The preparation of claim 18, wherein the preparation comprises from 40% to 80% by weight of water, based on a total weight of the preparation.

35. The preparation of claim 18, wherein the preparation comprises from 0% to less than 1% by weight, based on a total weight of the preparation, of polyethylene glycols and polyethylene glycol derivatives.

36. A method of improving the stability and/or sensorics of a W/O emulsion, wherein the method comprises including in the emulsion two W/O emulsifiers which differ in their HLB value by at most 1.

37. The method of claim 36, wherein the two W/O emulsifiers are diisostearoyl polyglyceryl-3 dimer dilinoleate and polyglyceryl-4 diisostearate/polyhydroxystearate/sebacate.
